(19) United States
(12) Patent Application Publication
(10) Pub. No.: US 2021/0196140 A1
(43) Pub. Date: Jul. 1, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Kosuke AOKI, Kanagawa (JP); Tadashi SUTO, Kanagawa (JP); Tsutomu KIMURA, Kanagawa (JP)

(73) Assignee: FUJI XEROX CO., LTD., Tokyo (JP)

(21) Appl. No.: 16/893,979

(22) Filed: Jun. 5, 2020

(30) Foreign Application Priority Data
Dec. 27, 2019 (JP) 2019-238648

(51) Int. Cl. A61B 5/04; A61B 5/0476; A61B 5/0488; A61B 5/00; A61B 5/16; G16H 40/63

(52) U.S. Cl. CPC A61B 5/04012; A61B 5/0476; A61B 5/0488; A61B 5/1126; A61B 5/165; G16H 40/63; A61B 2562/06; A61B 5/6817

(57) ABSTRACT

An information processing apparatus includes a processor. The processor is configured to acquire, from a biometric potential acquired from a subject, first information representing a feeling of the subject and second information representing a movement of a body that the subject consciously takes, and output, in an associated form, the first information and the feeling of the subject pre-associated with the second information.

### CROSS-REFERENCE TO RELATED APPLICATIONS

This application is based on and claims priority under 35 USC 119 from Japanese Patent Application No. 2019-238648 filed Dec. 27, 2019.

### BACKGROUND

### (i) Technical Field

The present disclosure relates to an information processing apparatus and a non-transitory computer readable medium.

### (ii) Related Art

Japanese Unexamined Patent Application Publication No. 2018-68510 discloses a program causing a computer to execute a process. The process includes acquiring an electroencephalogram (EEG) signal that is measured via electrodes placed on multiple locations on the head of a subject, converting the acquired EEG signal into a frequency spectrum, extracting EEG components on a per frequency band basis from the frequency spectrum, determining whether the EEG components in an alpha band or the EEG components in a theta band are synchronized with each other in brain regions corresponding to the multiple locations, and determining whether the subject is wakeful, in accordance with the EEG signal or a EEG component in a frequency band different from a frequency band of the EEG component serving as a synchronization determination target.

Japanese Unexamined Patent Application Publication No. 2015-54240 discloses a content assessment system. The content assessment system includes an EEG measuring unit, a biological signal measuring unit, a sensor controller, and a controller. The EEG measuring unit measures an EEG of a subject who receives the delivery of content and outputs the EEG. The biological signal measuring unit measures a biological signal of the subject who receives the delivery of the content and outputs the biological signal. The sensor controller receives and transfers the EEG and biological signal by controlling the EEG measuring unit and biological signal measuring unit. The controller detects a change in the EEG and biological signal occurring prior to and during the delivery of the content by analyzing the EEG and biological signal transferred from the sensor controller, derives a degree of immersion direction and/or an emotion direction of the subject in the content by using the change in the EEG and biological signal, and assesses the content using at least one of derived results.

Japanese Patent No. 6423657 discloses an EEG signal analysis result display apparatus. The EEG signal analysis result display apparatus includes head electrodes, noise remover, specific band signal acquisition unit, a root-mean-square voltage determination unit, and analyzer. The head electrodes are placed on the head of a subject. The noise remover removes a noise component from an EEG signal obtained by the head electrodes via a noise removal technique as appropriate. The specific band signal acquisition unit acquires a specific band component signal from a low-artifact signal with noise removed therefrom. The root-mean-square voltage determination unit determines a root-mean-square voltage of the specific band signal. The analyzer displays on a display, in a two-dimensional graph with one axis representing the right hemisphere and the other axis representing the left hemisphere of the brain of the subject, a plot of the ensemble mean of analysis results of the left and right hemispheres. The ensemble mean of the analysis results of the left and right hemispheres is obtained by analyzing the time series signals of the root-mean-square voltages of the left and right hemispheres of the brain of the subject.

Estimating the feeling of the subject in accordance with a bioelectric potential representing the state of the body of the subject, such as electroencephalogram (EEG), has been studied.

A typical change in the bioelectric potential responsive to the feeling of the subject does not necessarily appear and it may be difficult to estimate the feeling of the subject from the bioelectric potential. There may be a time lag between the feeling of the subject and a change occurring in the bioelectric potential in response to the feeling. This also leads to the difficulty of estimating which time point the subject has had the feeling estimated from the bioelectric potential.

### SUMMARY

Aspects of non-limiting embodiments of the present disclosure relate to providing an information processing apparatus and a non-transitory computer readable medium to more accurately analyze the feeling of a subject than when the feeling of the subject is analyzed from bioelectric potential in a manner free from combining bioelectric information that the subject has consciously created.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus. The information processing apparatus comprising a processor configured to acquire, from a biometric potential acquired from a subject, first information representing a feeling of the subject and second information representing a movement of a body that the subject consciously takes and output, in an associated form, the first information and the feeling of the subject pre-associated with the second information.

### BRIEF DESCRIPTION OF THE DRAWINGS

Exemplary embodiment of the present disclosure will be described in detail based on the following figures, wherein:

FIG. 1 illustrates a configuration of an information processing system;

FIG. 2 illustrates a shape of a device in the system;

FIG. 3 is a graph of an example of a waveform of a bioelectric potential;

FIG. 4 illustrates decomposed bioelectric potentials;

FIG. 5 illustrates an example of a feeling table;

FIG. 6 is an electrical block diagram of an information processing apparatus;

FIG. 7 is a flowchart illustrating an example of a feeling analysis process;

FIGS. 8A and 8B illustrate an example of model waveforms of a myoelectric potential;

FIG. 9 illustrates an association example between an electroencephalogram (EEG) and feelings;

FIG. 10 illustrates an association example between multiple types of EEGs and subjective feelings;

FIG. 11 illustrates an example of a feeling table including operation instructions;

FIG. 12 illustrates a cancel operation for a subjective feeling; and

FIG. 13 illustrates a recovery operation for the subjective feeling.

### DETAILED DESCRIPTION

Exemplary embodiment of the disclosure is described with reference to the drawings. The same elements and operations are designated with the same reference numerals and the discussion thereof is not duplicated.

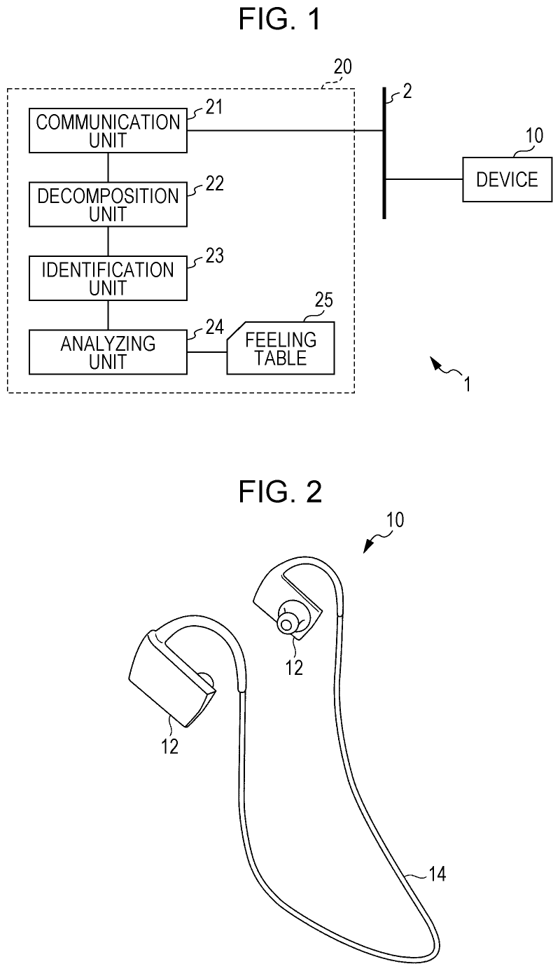

FIG. 1 illustrates the configuration of an information processing system 1 of the exemplary embodiment. The information processing system 1 includes a device 10 and information processing apparatus 20. The device 10 and the information processing apparatus 20 are connected to each other via a communication network 2.

The device 10 measures a bioelectric potential caused by life activity of human. The bioelectric potential includes a variety types. The types of the bioelectric potential include an electroencephalogram (EEG) representing the active state of a brain, myoelectric potential representing the activity state of muscle fibers, visual evoked potential representing the excited condition of optical nerves, and auditory evoked potential representing the excited condition of auditory nerves.

FIG. 2 illustrates the shape of the device 10. For example, the device 10 may have an earphone configuration. The device 10 includes sensor units 12 measuring the bioelectric potential and connected via a cable 14. When projections of the sensor units 12 are inserted into the ear canals of a person (hereinafter referred to as a subject) serving as a measurement target of the bioelectric potential, the bioelectric potential appearing in the inner ears of the subject is measured.

The sensor unit 12 in the device 10 includes not only the sensor measuring the bioelectric potential but also a six-axis sensor that measures a moving direction, moving speed, and acceleration of the head of the subject. The sensor unit 12 in the device 10 thus has a function of measuring the movement of the head of the subject. The sensor units 12 in the device 10 further includes a microphone that converts the voice of the subject into an electrical signal and outputs the electrical signal and a speaker that emits to the subject a voice instruction related to the measurement of the bioelectric potential.

As long as the device 10 includes at least the sensor measuring the bioelectric potential of the subject, the device 10 may additionally include other sensors and functions. The shape of the device 10 may not necessarily be of an earphone type. There is no restriction of the measurement target location of the bioelectric potential by the device 10. The device 10 may acquire the bioelectric potential from any part of the body of the subject.

If the bioelectric potentials caused by the activity of organs in the head, such as electroencephalogram (EEG), visual evoked potential, and/or auditory evoked potential, are to be accurately measured, the sensor unit 12 in the device 10 is set to be closer to an organ serving as a measurement target that generates the bioelectric potential. To this end, the sensor unit 12 in the device 10 is desirably mounted on the head. In accordance with the exemplary embodiment, the sensor units 12 in the device 10 are inserted into the ear canals of the subject to measure the bioelectric potentials including the EEG.

The EEG of the subject represents a latent feeling of the subject. Given the same feeling, the appearing EEG may be different from subject to subject.

The information on a movement responsive to the type of each feeling (hereinafter referred to as a motion) is conveyed to the subject such that the subject is able to indicate his or her feeling he or she has during the measurement of the bioelectric potential. If the subject has a feeling during the measurement of the bioelectric potential, he or she indicates the feeling by consciously taking the corresponding motion. The myoelectric potential varies depending on the type of motion. A change in the myoelectric potential occurs in response to the motion of the subject indicating the feeling. The feelings are thus more easily analyzed. The latent feeling is a feeling which the subject is not aware of having.

In the following discussion, a feeling consciously expressed via the motion by the subject is referred to a “subjective feeling” and a latent feeling of the subject represented by the EEG is referred to as a “latent feeling”. The EEG representing the latent feeling is an example of first information in the exemplary embodiment and the myoelectric potential of the subject represents the motion of the subject and is thus an example of second information in the exemplary embodiment.

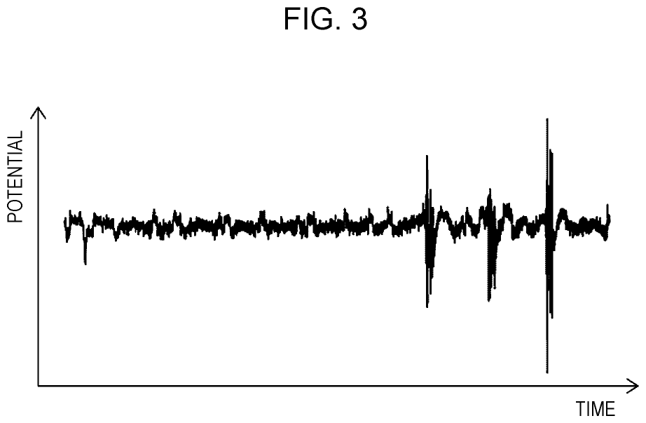

The device 10 transmits a measured bioelectric potential to the information processing apparatus 20 via the communication network 2. FIG. 3 is a graph plotting an example of a waveform of the bioelectric potential measured by the device 10. Referring to FIG. 3, the horizontal axis of the graph represents time and the vertical axis of the graph represents potential.

There are various types of bioelectric potentials. Since individual potentials do not separately appear on the body of the subject, the device 10 measures the bioelectric potential on which a variety of types of potentials are superimposed.

The information processing apparatus 20 has functions for a communication unit 21, decomposition unit 22, identification unit 23, and analyzing unit 24 and a feeling table 25. The communication unit 21 receives via the communication network 2 the bioelectric potential of the subject measured by the device 10.

The decomposition unit 22 decomposes the bioelectric potential of the subject received by the communication unit 21 into multiple types of pre-superimposed bioelectric potentials.

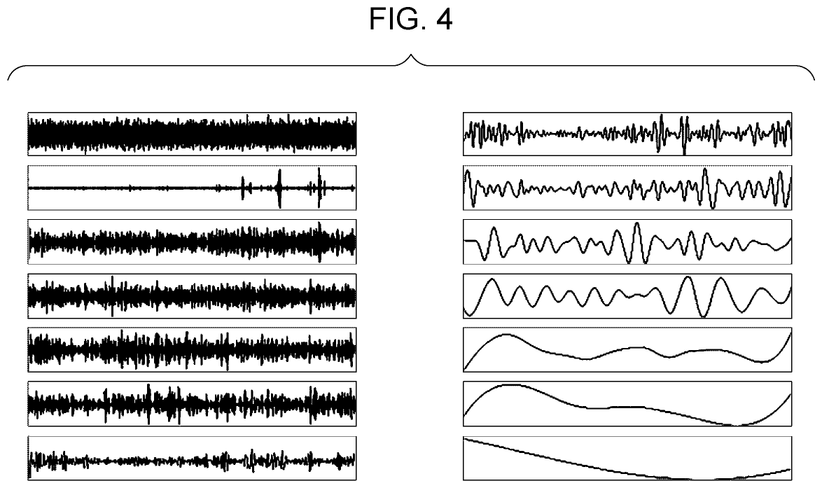

FIG. 4 illustrates the bioelectric potential in FIG. 3 into multiple types of bioelectric potentials. At this time point, the type indicated by a decomposed bioelectric potential is not yet determined.

The identification unit 23 identifies the bioelectric potential indicating the myoelectric potential of the subject and noise (also referred to as a noise component) from the bioelectric potentials decomposed by the decomposition unit 22. For example, the myoelectric potential contains a frequency component characteristic of the myoelectric potential and the noise contains a frequency component characteristic of the noise. Referring to a difference in the frequency component characteristic of the type of bioelectric potential, the identification unit 23 identifies from the decomposed bioelectric potentials the myoelectric potential of the subject and the bioelectric potential representing the noise.

The identification unit 23 identifies a specific motion of the subject by referring to a change (hereinafter referred to as a “myoelectric potential waveform”) along the time series of the myoelectric potential of the subject.

The analyzing unit 24 refers to the feeling table 25 that pre-associates the motion of the subject with the feeling of the subject and analyzes the myoelectric potential waveform to determine what feeling the subject has expressed during what time period.

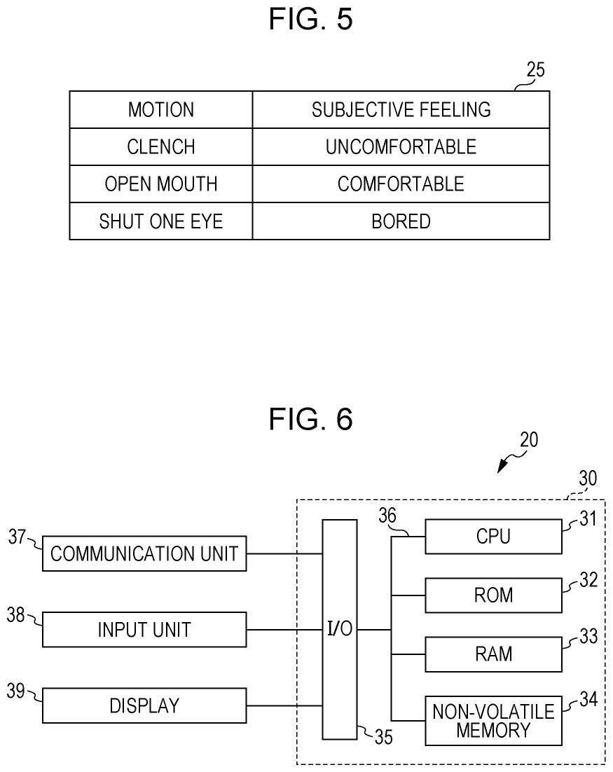

FIG. 5 illustrates an example of the feeling table 25. In the feeling table 25 in FIG. 5, the motion of the subject is associated with the subjective feeling. The feeling table 25 indicates that if the subject clenches, the subject may possibly feel uncomfortable. The feeling table 25 also indicates that if the subject opens his or her mouth, the subject may possibly feel comfortable. The feeling table 25 indicates that if the subject shuts one eye, the subject may possibly feel bored.

The feeling table 25 in FIG. 5 is only an example of feeling table. The feeling table 25 associates a variety of subjective feelings respectively with motions. The subject may express a subjective feeling by taking a motion defined in the feeling table 25.

The analyzing unit 24 associates the analyzed subjective feeling of the subject with the EEG in a time band in which the subjective feeling has appeared, and the analyzing unit 24 comprehensively analyzes the feeling of the subject in accordance with a change state in the EEG and the subjective feeling of the subject.

Communication protocol used in the communication network 2 is not limited to any particular protocol. The communication network 2 may be a wired or wireless network. The communication network 2 may be an exclusive network or a network open to public, such as the Internet, which shares lines with unspecified large number of users.

In the information processing system 1 of the exemplary embodiment, the device 10 and the information processing apparatus 20 are connected via the communication network 2. The device 10 is not necessarily connected to the information processing apparatus 20 via the communication network 2. If the device 10 is not connected to the information processing apparatus 20 via the communication network 2, the bioelectric potential measured by the device 10 may be stored on a portable storage medium that is removable from the device 10 and the information processing apparatus 20 and the bioelectric potential is thus exchanged between the device 10 and the information processing apparatus 20 using the storage medium.

FIG. 6 is an electrical block diagram of the information processing apparatus 20. Referring to FIG. 6, the information processing apparatus 20 may be a computer 30.

The computer 30 includes a central processing unit (CPU) 31, read-only memory (ROM) 32, random-access memory (RAM) 33, non-volatile memory 34, and input and output (I/O) interface 35. The CPU 31 is an example of processor that performs an operation of each element in the information processing apparatus 20 in FIG. 1. The ROM 32 stores an information processing program that causes the computer 30 to function as the information processing apparatus 20. The RAM 33 serves as a temporary memory region for the CPU 31. The CPU 31, ROM 32, RAM 33, non-volatile memory 34, and I/O interface 35 are mutually connected to each other via a bus 36.

The non-volatile memory 34 is an example of memory that maintains stored data even if power to the non-volatile memory 34 is shut down. For example, the non-volatile memory 34 is a semiconductor memory. A hard disk may also be used for the non-volatile memory 34. The non-volatile memory 34 may not necessarily be internal to the computer 30. The non-volatile memory 34 may be a universal serial bus (USB) memory or memory card that is portable and removable from the computer 30.

The I/O interface 35 is connected to a communication unit 37, input unit 38, and display 39.

The communication unit 37 is connected to the communication network 2 and supports a communication protocol in accordance with which data communication with an external apparatus connected to the device 10 and the communication network 2 is performed.

The input unit 38 receives an instruction from the user and notifies the CPU 31 of the instruction. For example, the input unit 38 may be a button, keyboard, and/or mouse. If the instruction is to be given in voice, a microphone may be used for the input unit 38.

The display 39 outputs information processed by the CPU 31. For example, the display 39 may be a liquid-crystal display, electroluminescent (EL) display, or projector.

Elements connected to the I/O interface 35 in the computer 30 are not limited to those in FIG. 6. For example, an image forming unit forming an image on a recording medium, such as a paper sheet, may be connected to the I/O interface 35 and print the feeling in text as analysis results provided by the CPU 31.

A feeling analysis process of the information processing apparatus 20 is described below in detail.

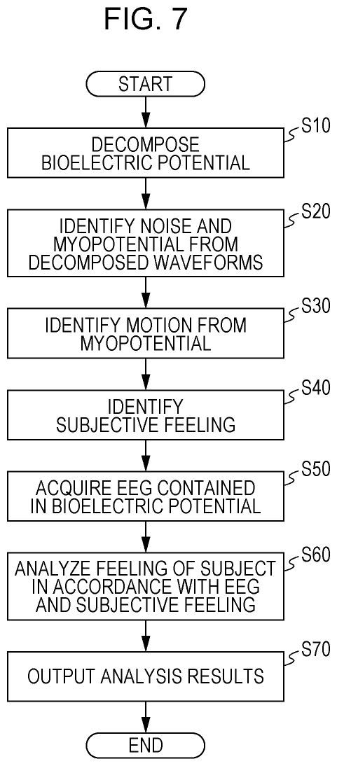

FIG. 7 is a flowchart illustrating the feeling analysis process performed by the CPU 31 in the information processing apparatus 20 when the information processing apparatus 20 receives the bioelectric potential of the subject measured by the device 10.

An information processing program defining the feeling analysis process is pre-stored on the ROM 32 in the information processing apparatus 20. The CPU 31 in the information processing apparatus 20 reads the information processing program from the ROM 32 and executes the feeling analysis process.

In step S10, the CPU 31 decomposes the bioelectric potential of the subject received from the device 10 into multiple types of pre-superimposed bioelectric potentials. Related art technique, such as empirical mode decomposition (EMD), may be used to decompose the bioelectric potential.

Based on the assumption that the waveform is represented by a sum of multiple basis functions even though the waveform of the bioelectric potential is not clear as to what basis function the bioelectric potential contains, EMO decomposes the waveform by estimating the basis function.

Specifically, let variable t represent time, x(t) a waveform as a decomposition target, and y(t) a single basis function in the waveform x(t), and the CPU 31 detects maximum and minimum points of the waveform by detecting all extreme values in the waveform x(t) (first operation).

The CPU 31 interpolates between the detected maximum and minimum points and determines an upper envelope emax(t) that connects the maximum points and a lower envelope emin(t) that connects the minimum points (second operation). The CPU 31 calculates a local average m(t) of the upper envelope emax(t) and the lower envelope emin(t) in accordance with equation (1) (third operation).

$$m ( t ) = \frac{e max ( t ) + e min ( t )}{2} \qquad ( 1 )$$

The CPU 31 treats as a new x(t) a difference waveform yᵣ(t) represented by a difference between the waveform x(t) and the local average m(t), namely, Yr(t)=x(t)−m(t) (fourth operation) and repeats the first through fourth operations until the difference waveform yᵣ(t) falls to or below a predetermined value (fifth operation).

The CPU 31 sets to be the basis function y(t) the difference waveform yᵣ(t) that is equal to or below the predetermined value.

The CPU 31 further acquires another basis function using the basis function y(t) thus acquired. Specifically, the CPU 31 treats as a new waveform x(t) a difference x₁(t) between the waveform x(t) and the waveform y(t), namely, x₁(t)=x(t)−y(t) (sixth operation). The CPU 31 repeats the first through sixth operations. The CPU 31 ends the acquisition of the basis function y(t) when xₙ(t) having a single extreme value is obtained.

The original waveform x(t) is decomposed into the waveforms represented by the n basis functions y(t) (n is the number of iterations of the first through six operations). In the following discussion, the waveforms of the bioelectric potential represented by the basis functions y(t) are referred to as “decomposed waveforms”.

In step S20, the CPU 31 performs the Fourier transform on the decomposed waveforms acquired in step S10 to obtain frequency spectra. Based on a frequency component characteristic of the types of bioelectric potentials and the intensity of the frequency component, the CPU 31 identifies from the decomposed waveform the myoelectric potential waveform of the subject and waveform representing noise. A frequency attribute value representing a combination of frequency components characteristic of the myoelectric potential and noise and the intensity of the frequency components is pre-stored on the non-volatile memory 34. The CPU 31 reads from the non-volatile memory 34 the frequency attribute value responsive to the waveform of each bioelectric potential and calculates a similarity of the frequency attribute value to the frequency spectrum of the decomposed waveforms. The CPU 31 thus identifies from the decomposed waveforms the waveforms representing the myoelectric potential of the subject and the noise. Since the myoelectric potential and the noise are separately identified, the noise is removed from the myoelectric potential waveform of the subject.

The myoelectric potential of the subject tends to have a larger amount of change in amplitude per unit time than the EEG and the noise tends to be continuously changing in amplitude. The CPU 31 may identify the waveforms representing the myoelectric potential of the subject and the waveform representing the noise, in accordance with the characteristics of the change in amplitude or a combination of the characteristic of the change in amplitude and the frequency attributes.

In step S30, the CPU 31 retrieves from the non-volatile memory 34 a model waveform typical of the myoelectric potential measured by the device 10 in response to each motion and compares the model waveform of the myoelectric potential with a myoelectric potential waveform of the subject identified in step S20. If the myoelectric potential waveform of the subject has a portion where a waveform similar to the model waveform of any myoelectric potential measured has appeared, the CPU 31 determines that the subject has taken, in a time band during which the waveform similar to the model waveform of the myoelectric potential has appeared, the motion represented by the similar model waveform of the similar myoelectric potential.

A determination as to whether the model waveform of the myoelectric potential is similar to the myoelectric potential waveform of the subject is performed using the similarity determination technique of the related art, such as pattern recognition of waveforms. The similarity determination technique used in the exemplary embodiment is based on the assumption that a larger degree of similarity is output as the model waveform of the myoelectric potential is more similar to the myoelectric potential waveform of the subject. The CPU 31 thus determines that at a location having a similarity of a predetermined threshold value or more, the subject has taken the motion represented by the model waveform of the myoelectric potential having the similarity of the threshold value or more.

The CPU 31 may identify the motion performed by the subject in accordance with the similarity between the frequency spectrum of the model waveform of the myoelectric potential and the frequency spectrum of the myoelectric potential waveform of the subject.

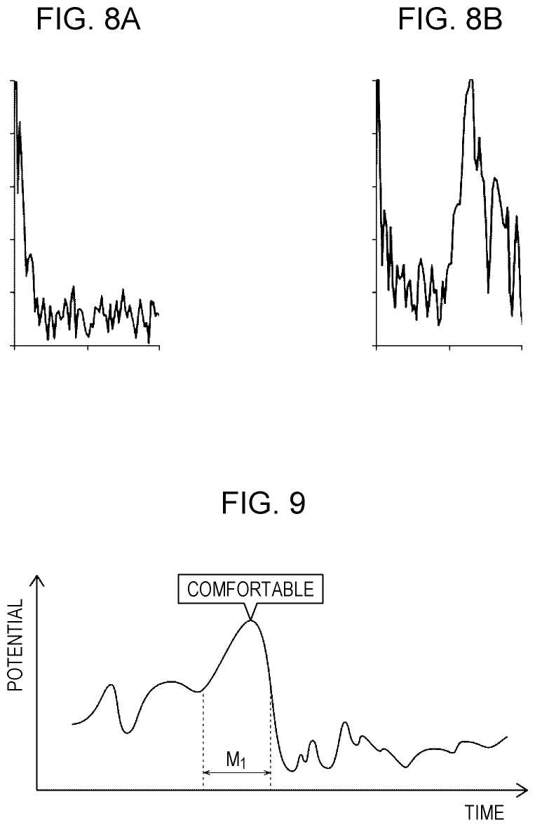

FIGS. 8A and 8B illustrate examples of the model waveform of the myoelectric potential pre-stored on the non-volatile memory 34. FIG. 8A illustrates the example of the model waveform of the myoelectric potential appearing when the subject opens his or her mouth and FIG. 8B illustrates the example of the model waveform of the myoelectric potential appearing when the subject clenches.

The person that the model waveform of the myoelectric potential is acquired from is not limited to any particular person but even given the same motion, the waveform of the myoelectric potential may be different from person to person. Before a feeling analysis process, the user has the subject perform each motion listed in the feeling table 25 and the myoelectric potential corresponding to the motion is desirably compared as a model waveform with the myoelectric potential waveform of the subject identified in step S20.

The model waveform of the myoelectric potential may not necessarily be stored on the non-volatile memory 34 and may be stored on an external device, such as a data server, which may use a cloud connected to the communication network 2. The memory capacity available in the cloud may be increased as appropriate. For example, if the computer 30 including the implemented non-volatile memory 34 that is limited in memory capacity is a mobile terminal, such as a smart phone, the CPU 31 may refer to model waveforms in larger number than the model waveforms available on the non-volatile memory 34 in the mobile terminal.

In step S40, the CPU 31 refers to the feeling table 25 and identifies the subjective feeling of the subject corresponding to the motion and the time band in which the subjective feeling has appeared. The CPU 31 identifies the subjective feeling and time band in accordance with the contents of the motion identified in step S30 and the location of occurrence of the motion in the myoelectric potential waveform of the subject.

In step S50, the CPU 31 acquires the EEG of the subject. Specifically, the CPU 31 superimposes the remaining waveforms decomposed in step S10 other than the waveform of the myoelectric potential of the subject and the waveform representing noise and sets the superimposed waveform to be the EEG.

In step S60, the CPU 31 associates the subjective feeling of the subject identified in step S40 with the EEG having appeared in the time band in which the subject has expressed the subjective feeling. The CPU 31 comprehensively analyzes the feeling of the subject in accordance with the latent feeling indicated by the EEG of the subject and the subjective feeling of the subject.

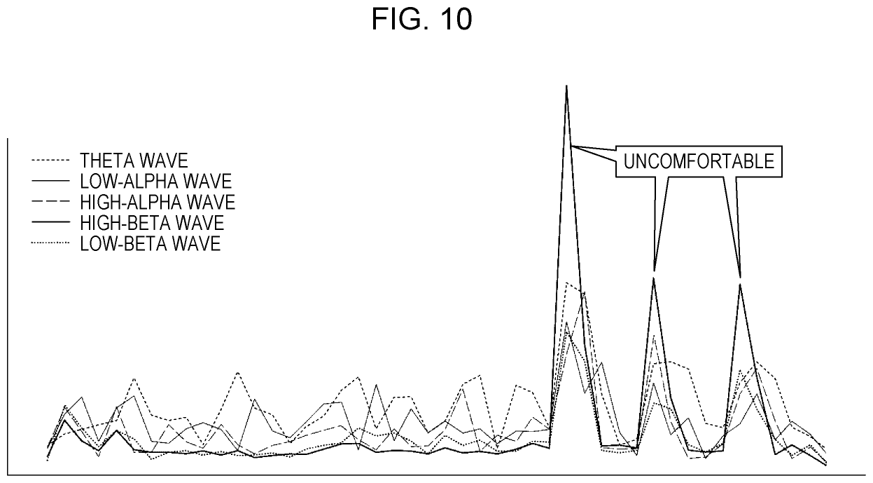

FIG. 9 illustrates an association example between the EEG of the subject and the subjective feeling of the subject. Referring to FIG. 9, the myoelectric potential waveform corresponding to the motion of opening the mouth is confirmed in a period M₁, the EEG appearing in the period M₁ is associated with the subjective feeling “comfortable” in accordance with the feeling table 25 in FIG. 5.

The CPU 31 performs gap analysis and recognition analysis to analyze the feeling of the subject. The gap analysis is performed to determine, in accordance with state information that associates the EEG of the subject with the subjective feeling of the subject, whether the subjective feeling of the subject has shifted from the latent feeling of the subject determined from the EEG. The recognition analysis is performed to recognize a time lag for the subject to recognize the subject's own feeling from the occurrence of the latent feeling and to consciously take the motion in response to the feeling. The CPU 31 may not necessarily have to analyze the feeling of the subject in accordance with the state information that associates the EEG of the subject with the subjective feeling of the subject and may simply associate the EEG of the subject with the subjective feeling of the subject. Associating the EEG of the subject with the subjective feeling of the subject is an example of feeling analysis.

In step S70, the CPU 31 outputs analysis results of the subject obtained in step S60 from the information processing apparatus 20 and then displays the analysis results on the display 39. As long as the analysis results are output from the information processing apparatus 20, the manner of outputting the analysis results is not limited to any particular way. For example, the analysis results may be printed on a recording medium on an image forming unit (not illustrated) connected to the I/O interface 35 or a network printer (not illustrated) connected to the communication network 2. Data indicative of the analysis results may be stored on a data server (not illustrated) connected to the communication network 2. Another apparatus different from the information processing apparatus 20 may further analyze the feeling of the subject using the analysis results stored on the data server.

The feeling analysis process in FIG. 7 is thus complete. The association between the EEG of the subject and the subjective feeling in FIG. 9 may be the analysis results of the feeling of the subject who has watched a television program or has listened to a radio program. The analysis results may help a user to know a scene the subject has expressed a feeling at and may serve as reference materials in producing programs. The feeling analysis process of the information processing apparatus 20 may be applicable to the examination of the brain function of the subject or psychological examination of the subject.

Through the feeling analysis process, the information processing apparatus 20 may analyze which scene the subject feels comfortable at and may provide service to the subject. For example, the information processing apparatus 20 may advise the subject of how to change his or her mind or notify the subject that the subject is in a suitable physical and mental condition for study or work.

Through the feeling analysis process, the information processing apparatus 20 decomposes a single bioelectric potential measured by the device 10 into the EEG, myoelectric potential, and noise. The burden on the subject is thus small in comparison with the case in which different types of sensors, including a sensor measuring the myoelectric potential, a sensor measuring the EEG, and noise sensor, are mounted on the subject. If the bioelectric potentials are measured using different types of sensors, a preprocess is to be performed. The preprocess may include unit conversion on a per bioelectric potential basis, time axis alignment, and missing data interpolation. In the feeling analysis process of the exemplary embodiment, time for the feeling analysis is saved in comparison with the case in which the feeling of the subject is analyzed with the bioelectric potentials measured using different types of sensors for different types of bioelectric potentials.

The user has the subject take the motion during the measurement of the bioelectric potential. Alternatively, after the measurement, the user has the subject remember the feeling he or she has had during the measurement of the bioelectric potential and fill out a questionnaire. The subjective feeling of the subject is thus obtained. However, since time has elapsed since the occurrence of the feeling, the feeling during the measurement may not be correctly written.

If the subject is made to fill out the questionnaire about the feeling during the measurement, the subject may pay attention to writing and the bioelectric potential prior to and subsequent to the writing may not correctly represent the feeling of the subject.

The feeling analysis process of the exemplary embodiment may accurately analyze the feeling of the subject in comparison with the case in which the subjective feeling of the subject is acquired by having the subject to fill out the questionnaire about the feeling.

In step S50 of the feeling analysis process in FIG. 7, the EEG of the subject is acquired by superimposing all the waveforms, excluding waves representing the myoelectric potential of the subject and noise. The EEG includes multiple types of waves.

The EEG is divided into theta wave, alpha wave, and beta wave. The theta wave is generated when human is in a quiet state, for example, when human is dozing and shifting from an awake state to a sleep state. Insight and inspiration are more easily activated in the theta wave state than in other state. The alpha wave is generated when human is relaxed. In the alpha-wave state, concentration and memory are better than in other state. The beta wave is generated when human is nervous or anxious. The beta-wave state indicates that human is awaking.

Each type of the EEG has its own particular frequency range. The theta wave is in a frequency range of 4 Hz or higher and lower than 8 Hz. The alpha wave is in a frequency range of 8H or higher and lower than 14 Hz. The beta wave is in a frequency range of 14 Hz or higher or lower than 30 Hz. If pre-superimposed waveforms contain a waveform having the same frequency range as the frequency range of any type of the EEG, that waveform represents the same type of the EEG.

The CPU 31 may identify the waveform corresponding to each type of the EEG from the frequency spectrum of the waveform decomposed in step S20 in FIG. 7. In step S50 in FIG. 7, the CPU 31 superimposes waveforms, excluding waveforms representing the myoelectric potential of the subject and noise and a waveform corresponding to an identified EEG. The CPU 31 acquires an EEG that has not yet been identified as a specific type.

FIG. 10 illustrates an association example in which the type of the EEG is associated with the subjective feeling of the subject by identifying the type of the EEG appearing on the subject. Relationship between the feeling of the subject and a change in the EEG is identified by decomposing the EEG of the subject according to type and associating the type with the subjective feeling. Relatively more accurate results concerning the feeling of the subject are obtained than when the feeling of the subject is analyzed by associating the single EEG with the subjective feeling as illustrated in FIG. 9.

Referring to FIG. 10, the alpha wave is divided into low-alpha wave and high-alpha wave and the beta wave is divided into low-beta wave and high-beta wave. The low-alpha wave is in a frequency range of 8 Hz or higher and lower than 11 Hz. The high-alpha wave is in a frequency range of 11 Hz or higher and lower than 14 Hz. The low-beta wave is in a frequency range of 14 Hz or higher and lower than 22 Hz. The high-beta wave is in a frequency range of 22 Hz or higher and lower than 30 Hz.

The analysis results as a record of the association between the EEG of the subject and the subjective feeling of the subject (hereinafter referred to as association analysis results) are accumulated as illustrated in FIGS. 9 and 10. If a change in the EEG is then analyzed, expression tendency of the subjective feeling indicating what feeling the subject expresses at what position is thus obtained.

If the number of association analysis results accumulated reaches a predetermined number, the CPU 31 identifies the subjective feeling from the EEG of the subject in accordance with the past accumulated association analysis results, rather than identifying the subjective feeling of the subject from the myoelectric potential waveform. The CPU 31 thus associates the EEG of the subject with the subjective feeling. Specifically, the CPU 31 inputs the EEG of the subject to an estimation model. The estimation model is obtained by machine-learning as learning data an association between the subjective feeling and a change in the EEG in the accumulated association analysis results. The CPU 31 thus simply associates a feeling, which the estimation model has output in response to the change in the input EEG, with the EEG input as the subjective feeling at the location of the change in the EEG.

Once the number of association analysis results has reached the predetermined number, the subject is free from taking the motion expressing the subjective feeling and the information processing apparatus 20 estimates the subjective feeling of the subject from the EEG and associates the subjective feeling with the EEG. In such a case, the CPU 31 is free from performing the operation in step S30 to identify the motion of the subject from the myoelectric potential waveform of the subject and the operation in step S40 to identify the subjective feeling of the subject from the feeling table 25 in the feeling analysis process in FIG. 7.

The predetermined number of association analysis results is set to be the number of association analysis results used to estimate at a specified accuracy level the subjective feeling of the subject from the EEG of the subject.

As an example, the subjective feeling of the subject is estimated from the EEG of the subject when the number of accumulated association analysis results of the subject reaches the predetermined number. Alternatively, the subjective feeling of the subject is estimated from the EEG of the subject when the number of measurements of the bioelectric potential of the subject during unit time, for example, 1 month, reaches a predetermined number or more.

As described above, the feeling of the subject is analyzed by associating the subjective feeling with the EEG of the subject. Alternatively, the information processing apparatus 20 may analyze the feeling of the subject by acquiring from the measured the bioelectric potential a cardiac potential representing a pulse wave and by combining the acquired cardiac potential with the subjective feeling. The information processing apparatus 20 may also analyze the feeling of the subject by combining the acquired cardiac potential with the EEG and the subjective feeling.

The information processing apparatus 20 may analyze the feeling of the subject by acquiring the bioelectric potential that is obtained by measuring a skin potential and by combining the acquired skin potential with the subjective feeling. The skin potential changes in response to a resistance value of a body surface that changes in response to sweat secretion. The information processing apparatus 20 may analyze the feeling of the subject by combining the acquired skin potential and at least one of the EEG and the cardiac potential with the subjective feeling.

### Modifications

The subjective feeling is associated with the motion of the subject in the feeling table 25 in FIG. 5. Additionally, an operation instruction related to the association between the subjective feeling and the EEG may be also associated with the subjective feeling.

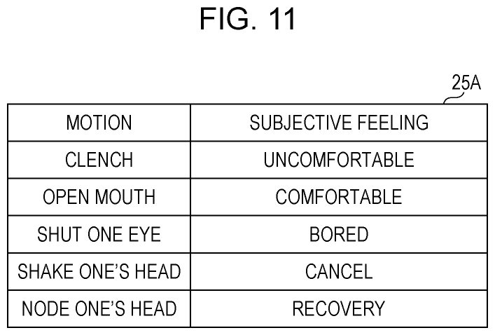

FIG. 11 illustrates a feeling table 25A further including the operation instruction related to the association between the subjective feeling and the EEG.

The feeling table 25A in FIG. 11 defines operations for the feeling association “cancel” and “recovery” that are not listed in the feeling table 25. For convenience of explanation, operations (motions) are defined for the subjective feeling in the feeling table 25A in FIG. 11.

The non-volatile memory 34 pre-stores the model waveforms of the myoelectric potentials corresponding to the motion of shaking head and the motion of nodding.

If a waveform similar to the model waveform of the myoelectric potential when the subject shakes head is recognized in the myoelectric potential waveform of the subject in step S30 of the feeling analysis process in FIG. 7, the CPU 31 determines that the subject gives an instruction to cancel the subjective feeling. Cancelling the subjective feeling is an operation that cancels the subjective feeling the subject expresses prior to the cancelling operation.

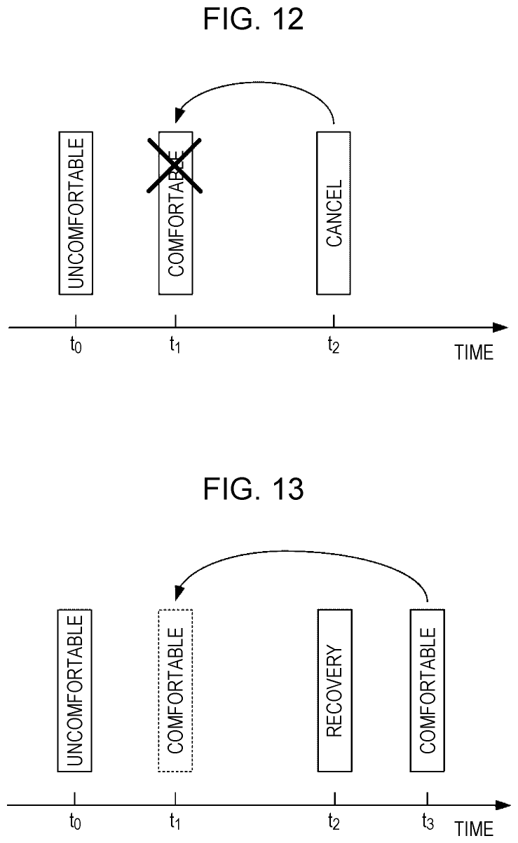

FIG. 12 illustrates the cancel operation to cancel the subjective feeling. Referring to FIG. 12, the subject expresses an uncomfortable feeling at time t₀, then expresses a comfortable feeling at time t₁, and cancels the comfortable feeling by shaking head at t₂. The CPU 31 cancels the subjective feeling the subject expresses prior to the cancel operation. The CPU 31 cancels the comfortable feeling as the subjective feeling immediately prior to the cancel operation. The cancel target may be set in advance. For example, the subjective feelings falling within a range extending back by N operations from the cancel operation (N is an integer equal to 1 or larger) may be canceled or the subjective feelings falling within a range extending back by a predetermined time period (cancel period) from the cancel operation at time t₂ may be canceled.

The cancel number N of the subjective feelings to be canceled by the cancel operation and the cancel period may be pre-stored on the non-volatile memory 34 and updated by an operation of a person in charge of feeling analysis.

The cancel number N of the subjective feelings to be canceled by the cancel operation and the cancel period may be requested by the subject, rather than being stored on the non-volatile memory 34. Referring to FIG. 12, if the subject takes the motion responsive to information “2” in succession to the cancel operation, the CPU 31 cancels the subjective feeling as “uncomfortable” at time t₀ and the subjective feeling as “comfortable” at time t₁. If the subject takes the motion responsive to information “5 minutes” in succession to the cancel operation, the CPU 31 cancels all the subjective feelings falling within the range extending back by 5 minutes from time t₂. The model waveforms of the myoelectric potential when the subject takes the motion responsive to the information indicating the value, such as “2” or “5 minutes”, are pre-stored on the non-volatile memory 34 or the like.

The subject may cancel the subjective feeling by taking the motion responsive to the information that specifically specifies the subjective feeling to be canceled, for example, “2 cycles earlier” or “10 minutes earlier”. Referring to FIG. 12, if the subject preforms the motion to cancel the subjective feeling 2 cycles earlier in succession to the cancel operation, the CPU 31 cancels the uncomfortable subjective feeling at time t₀, namely, the second subjective feeling 2 cycles earlier than the cancel operation at t2. In this case, the CPU 31 does not cancel the comfortable subjective feeling at time t₁. If the subject performs the motion to cancel the subjective feeling 10 minutes earlier in succession to the cancel operation, the CPU 31 cancels the subjective feeling indicated 10 minutes earlier than the cancel operation at t2. In such a case, even if a subjective feeling is displayed 5 minutes earlier, the CPU 31 does not cancel that subjective feeling.

The subject may cancel the subjective feeling within a range specified by a motion, after the cancel operation. For example, the motion may specify a range of from 2 cycles to 4 cycles earlier or a range of 10 minutes to 15 minutes earlier.

If a waveform similar to the model waveform of the myoelectric potential when the subject is nodding is recognized in the myoelectric potential waveform of the subject, the CPU 31 determines that the subject has requested a recovery of the subjective feeling. For example, even though the subject has a specific subjective feeling defined in the feeling table 25A during the measurement of the bioelectric potential, he or she may forget to take the motion corresponding to the subjective feeling. The recovery of the subjective feeling means an operation to recover the subjective feeling by retrospectively associating the motion with the subjective feeling at the time of occurrence.

FIG. 13 illustrates the recovery operation for the subjective feeling. Referring to FIG. 13, the subject expresses an uncomfortable feeling at time t₀ and has a comfortable feeling at time t₁ but forgets to perform the motion indicating the comfortable feeling, namely, the motion of opening mouth. The subject then shakes head at time t₂ to recover the subjective feeling.

The subjective feeling expressed by the subject first after a recovery operation is associated with a period prior to the recovery operation. Referring to FIG. 13, the subject expresses a comfortable feeling at t₃ after the recovery operation, and the CPU 31 associates the comfortable feeling as the subjective feeling with the period prior to the recovery motion. Specifically, the CPU 31 associates the comfortable feeling as the subjective feeling indicated first after the recovery operation with time (time t₁ in FIG. 13) that is predetermined time period (specified time period) earlier than the recovery operation at time t₂.

The specified time is pre-stored on the non-volatile memory 34 and may be modified by an operation by a person in charge of the feeling analysis.

The specified time may not necessarily be stored on the non-volatile memory 34 and may be entered by the motion of the subject. Referring to FIG. 13, if the subject indicates the subjective feeling after taking a motion corresponding to “3 minutes earlier” in succession to the recovery operation, the CPU 31 associates the subjective feeling, indicated first by the subject after the motion corresponding to the information indicating “3 minutes earlier”, with time that is 3 minutes earlier than the recovery operation at time t₂.

If another subjective feeling is associated with the time that is the specified time earlier than the recovery operation at time t₂, the CPU 31 may replace the associated subjective feeling with the subjective feeling that is indicated first after the recovery operation.

According to the modification of the exemplary embodiment, the subject may not only express the subjective feeling during the measurement of the bioelectric potential but also perform the operation related to the association between the subjective feeling and the EEG.

According to the exemplary embodiment, the feeling analysis process is implemented using software. The process in the flowchart in FIG. 7 may be performed using hardware, such as application specific integrated circuit (ASIC), field programmable gate array (FPGA), or programmable logic device (PLD). In such a case, a higher processing speed may be achieved than when the feeling analysis process is performed using software.

The CPU 31 may be a dedicated processor specialized in a particular process. The dedicated processor may be ASIC, FPGA, PLD, graphics processing unit (GPU), or floating point unit (FPU).

The process of the CPU 31 may be performed by one or more CPUs 31. The CPU 31 may perform the feeling analysis process in cooperation with another CPU 31 in the computer 30 that is at a physically separate location.

According to the exemplary embodiment, the information processing program is installed on the ROM 32. Alternatively, the information processing program may be supplied in a recoded form on a computer readable recording medium. The information processing program may be supplied in a recorded form on an optical disk, such as a compact disk (CD) ROM or digital versatile disk (DVD) ROM. The information processing program may be supplied in the recorded form on a portable semiconductor memory, such as a universal serial bus (USB) memory or memory card.

The information processing apparatus 20 may retrieve the information processing program via the communication unit 37 from an external apparatus connected to the communication network 2.

In the exemplary embodiment above, the term “processor” refers to hardware in a broad sense. Examples of the processor includes general processors (e.g., CPU: Central Processing Unit), dedicated processors (e.g., GPU: Graphics Processing Unit, ASIC: Application Specific Integrated Circuit, FPGA: Field Programmable Gate Array, and programmable logic device).

In the exemplary embodiment above, the term “processor” is broad enough to encompass one processor or plural processors in collaboration which are located physically apart from each other but may work cooperatively. The order of operations of the processor is not limited to one described in the exemplary embodiment above, and may be changed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The exemplary embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising a processor configured to
acquire, from a biometric potential acquired from a subject, first information representing a feeling of the subject and second information representing a movement of a body that the subject consciously takes, and
output, in an associated form, the first information and a feeling of the subject pre-associated with the second information.

2. The information processing apparatus according to claim 1, wherein the processor is configured to acquire an electroencephalogram of the subject as the first information from the bioelectric potential and acquire a myoelectric potential waveform of the subject as the second information from the bioelectric potential.

3. The information processing apparatus according to claim 2, wherein the processor is configured to
compare a model waveform of a myoelectric potential responsive to the movement of the body with the myoelectric potential waveform of the subject, and
associate the electroencephalogram of the subject in a time band during which the myoelectric potential waveform having a similarity of a predetermined value or above to the model waveform appears with a feeling of the subject pre-associated with the model waveform having the similarity of the predetermined value or above to the myoelectric potential waveform.

4. The information processing apparatus according to claim 3, wherein the processor is configured to,
if a number of analysis results that are records of association between the electroencephalogram of the subject and the feeling of the subject that is pre-associated with the movement represented by the model waveform is equal to or above a predetermined number,
associate the electroencephalogram of the user with the feeling of the subject using the analysis results without acquiring the myoelectric potential waveform of the subject from the bioelectric potential.

5. The information processing apparatus according to claim 3, wherein the model waveform is the myoelectric potential waveform that is responsive to the movement of the body and is acquired from the subject.

6. The information processing apparatus according to claim 4, wherein the model waveform is the myoelectric potential waveform that is responsive to the movement of the body and is acquired from the subject.

7. The information processing apparatus according to claim 3, wherein the processor is configured to compare the myoelectric potential waveform of the subject with the model waveform of the myoelectric potential responsive to the movement of the body after a noise component is removed from the bioelectric potential.

8. The information processing apparatus according to claim 4, wherein the processor is configured to compare the myoelectric potential waveform of the subject with the model waveform of the myoelectric potential responsive to the movement of the body after a noise component is removed from the bioelectric potential.

9. The information processing apparatus according to claim 5, wherein the processor is configured to compare the myoelectric potential waveform of the subject with the model waveform of the myoelectric potential responsive to the movement of the body after a noise component is removed from the bioelectric potential.

10. The information processing apparatus according to claim 6, wherein the processor is configured to compare the myoelectric potential waveform of the subject with the model waveform of the myoelectric potential responsive to the movement of the body after a noise component is removed from the bioelectric potential.

11. The information processing apparatus according to claim 1, wherein the feeling of the subject pre-associated with the second information includes an operation related to association of the feeling of the subject, and
wherein the processor is configured to,
if the second information is associated with the operation associated with the association of the feeling,
associate the feeling of the subject with the first information in accordance with contents of the operation associated with the second information.

12. The information processing apparatus according to claim 2, wherein the feeling of the subject pre-associated with the second information includes an operation related to association of the feeling of the subject, and
wherein the processor is configured to,
if the second information is associated with the operation associated with the association of the feeling,
associate the feeling of the subject with the first information in accordance with contents of the operation associated with the second information.

13. The information processing apparatus according to claim 3, wherein the feeling of the subject pre-associated with the second information includes an operation related to association of the feeling of the subject, and
wherein the processor is configured to, if the second information is associated with the operation associated with the association of the feeling, associate the feeling of the subject with the first information in accordance with contents of the operation associated with the second information.

14. The information processing apparatus according to claim 4, wherein the feeling of the subject pre-associated with the second information includes an operation related to association of the feeling of the subject, and
wherein the processor is configured to, if the second information is associated with the operation associated with the association of the feeling, associate the feeling of the subject with the first information in accordance with contents of the operation associated with the second information.

15. The information processing apparatus according to claim 5, wherein the feeling of the subject pre-associated with the second information includes an operation related to association of the feeling of the subject, and
wherein the processor is configured to, if the second information is associated with the operation associated with the association of the feeling, associate the feeling of the subject with the first information in accordance with contents of the operation associated with the second information.

16. The information processing apparatus according to claim 6, wherein the feeling of the subject pre-associated with the second information includes an operation related to association of the feeling of the subject, and
wherein the processor is configured to, if the second information is associated with the operation associated with the association of the feeling, associate the feeling of the subject with the first information in accordance with contents of the operation associated with the second information.

17. The information processing apparatus according to claim 11, wherein the processor is configured to,
if the contents of the operation associated with the second information represent cancelling of the feeling,
cancel the association performed, prior to the operation, between the feeling of the subject and the first information.

18. The information processing apparatus according to claim 11, wherein the processor is configured to,
if the contents of the operation associated with the second information represent a recovery from forgetting to express the feeling,
associate the feeling of the subject, acquired from the second information subsequent to the operation, with the first information during a period prior to the operation.

19. An information processing apparatus comprising processors means for:
acquiring, from a biometric potential acquired from a subject, first information representing a feeling of the subject and second information representing a movement of a body that the subject consciously takes; and
outputting, in an associated form, the first information and the feeling of the subject pre-associated with the second information.

20. A non-transitory computer readable medium storing a program causing a computer to execute a process for processing information, the process comprising:
acquiring, from a biometric potential acquired from a subject, first information representing a feeling of the subject and second information representing a movement of a body that the subject consciously takes; and
outputting, in an associated form, the first information and the feeling of the subject pre-associated with the second information.
